(19) United States
(12) Patent Application Publication
(10) Pub. No.: US 2021/0138149 A1
(43) Pub. Date: May 13, 2021

(54) ROLLING DIAPHRAGM SYRINGE WITH PISTON ENGAGEMENT PORTION

(71) Applicant: BAYER HEALTHCARE LLC, WHIPPANY, NJ (US)

(72) Inventors: Michael SPOHN, Fenelton, PA (US); Kevin COWAN, Allison Park, PA (US); Barry TUCKER, Verona, PA (US)

(21) Appl. No.: 17/059,787

(22) PCT Filed: May 30, 2019
(86) PCT No.: PCT/US2019/034556

§ 371 Date: Nov. 30, 2020

Related U.S. Application Data
(60) Provisional application No. 62/680,304, filed on Jun. 4, 2018.

(51) Int. Cl. A61M 5/142; A61M 5/00; A61M 5/315; A61M 5/145

(52) U.S. Cl. CPC A61M 5/14224; A61M 5/007; A61M 2005/14553; A61M 5/14566; A61M 5/31515

(57) ABSTRACT

A rolling diaphragm syringe has a proximal end having an end wall, a distal end, and a sidewall extending between the proximal end and the distal end. The rolling diaphragm syringe further has a piston engagement portion protruding proximally from a central portion of the end wall, the piston engagement portion having a central axis, and a plurality of grooves recessed inward relative to an outer surface of the piston engagement portion. The grooves are spaced apart from each other in a direction about the central axis. The piston engagement portion is configured for engagement with at least one engagement element of a piston of a fluid injector at least during movement of the piston in a proximal direction

### CROSS-REFERENCE TO RELATED APPLICATIONS

This application claims priority to U.S. Provisional Application No. 62/680,304, filed Jun. 4, 2018, the disclosure of which is hereby incorporated by reference in its entirety.

### BACKGROUND OF THE DISCLOSURE

### Field of the Disclosure

The present disclosure is related to syringes for use in the medical field and, more particularly, to syringes having a flexible sidewall and a piston engagement portion configured for engaging with an engagement mechanism of a piston of a fluid injector. The present disclosure particularly relate to configurations of a piston engagement portion of a syringe for improved engagement with engagement elements of a piston of a fluid injector.

### Description of Related Art

In many medical diagnostic and therapeutic procedures, a medical practitioner, such as a physician, injects a patient with one or more medical fluids. In recent years, a number of injector-actuated syringes and powered fluid injectors for pressurized injection of medical fluids, such as a contrast solution (often referred to simply as “contrast” or “contrast medium”), a flushing agent, such as saline, and other medical fluids, have been developed for use in procedures such as angiography, computed tomography (CT), ultrasound, magnetic resonance imaging (MRI), positron emission tomography (PET), and other imaging procedures. In general, these fluid injectors are designed to deliver a preset amount of fluid at a preset pressure and/or flow rate.

Typically, powered fluid injectors have at least one piston that is configured to connect to a plunger disposed within the syringe. The syringe generally includes a rigid barrel with the plunger being slidably disposed within the rigid barrel. The piston of the fluid injector drives the plunger in a distal direction relative to a longitudinal axis of the barrel to deliver the fluid from the syringe barrel and in the proximal direction to draw fluid into the syringe barrel.

Syringes used in the medical field are typically disposable and are discarded after one use. Although disposable syringes are typically made by mass production methods such as injection molding, such disposable syringes are relatively expensive due to the type and quantity of materials and precision involved in their manufacture, and the economic costs associated with packaging, shipping, and storage. Accordingly, it remains desirable to develop improved designs of syringes to facilitate injection procedures.

### SUMMARY OF THE DISCLOSURE

The present disclosure generally relates to syringes for use in the medical field and to methods of forming and using such syringes. The syringes may be useful in fluid delivery applications. The present disclosure further relates to fluid injectors having an engagement mechanism for engaging a piston engagement portion of a syringe.

In some embodiments of the present disclosure, a rolling diaphragm syringe may have a proximal end having an end wall, a distal end having an open-ended discharge neck, a sidewall extending between the proximal end and the distal end along a longitudinal axis, and a piston engagement portion protruding proximally from a central portion of the end wall. The piston engagement portion may have a central axis, and a plurality of grooves recessed inward relative to an outer surface of the piston engagement portion and spaced apart from each other in a direction about the central axis. At least a portion of the sidewall may be flexible such that the sidewall rolls upon itself with an outer surface of the sidewall at a folding region being folded in a radially inward direction when acted upon by an external force in a direction from the proximal end toward the distal end. The sidewall may be further configured to unroll such that the outer surface of the sidewall at the folding region is unfolded in a radially outward direction when acted upon by the external force in a direction from the distal end toward the proximal end.

In some embodiments of the present disclosure, the plurality of grooves may be spaced apart at equal or unequal intervals along the outer surface of the piston engagement portion in the direction about the central axis. The plurality of grooves may have equal or unequal width measured along the outer surface of the piston engagement portion in the direction about the central axis. The plurality of grooves may have equal or unequal depth measured from the outer surface of the piston engagement portion in a direction toward the central axis. Each groove may have a base and a pair of side surfaces extending from the base to the outer surface of the engagement portion. The plurality of grooves may be continuous over a longitudinal length of the piston engagement portion in a direction along the central axis. At least one of the plurality of grooves may be discontinuous over a longitudinal length of the piston engagement portion in a direction along the central axis.

In some embodiments of the present disclosure, an outer diameter of the piston engagement portion may be uniform or non-uniform in a direction along the central axis. An outer diameter of the piston engagement portion may decrease from a distal end of the piston engagement portion toward a proximal end of the piston engagement portion. In other embodiments, the outer diameter of the piston engagement portion may be greater at the distal and proximal ends along the longitudinal axis than at the middle of the piston engagement portion. In other embodiments, the outer diameter of the piston engagement portion may be greater at the proximal end than along the rest of the longitudinal axis of the piston engagement portion. The piston engagement portion may be monolithically formed with the end wall.

In some embodiments of the present disclosure, the piston engagement portion may be configured for engagement with at least one engagement element of a piston of a fluid injector at least during movement of the piston in a proximal direction. At least a portion of the piston engagement portion may be plastically or elastically deformed when engaged with the at least one engagement element of the piston. The piston engagement portion may have a widened portion at a proximal end, and the plurality of grooves may be recessed at least into the widened portion. The end wall may be concave and have a continuously increasing thickness in a direction toward the longitudinal axis. The central axis of the piston engagement portion may be coaxial with the longitudinal axis. The rolling diaphragm syringe may be in a first state where the end wall is inverted inwardly and rolled toward the distal end such that an internal volume of the rolling diaphragm syringe may be empty of fluid.

In some embodiments of the present disclosure, a syringe assembly for a fluid delivery system may have a pressure jacket having a pressure jacket distal end, a pressure jacket proximal end, and a throughbore extending between the pressure jacket distal end and the pressure jacket proximal end along a longitudinal axis. The syringe assembly further may have a rolling diaphragm syringe disposed within the throughbore of the pressure jacket. The rolling diaphragm syringe may have a proximal end having an end wall, a distal end having an open-ended discharge neck, a sidewall extending between the proximal end and the distal end along a longitudinal axis, and a piston engagement portion protruding proximally from a central portion of the end wall. The piston engagement portion may have a central axis and a plurality of grooves recessed inward relative to an outer surface of the piston engagement portion and spaced apart from each other in a direction about the central axis. At least a portion of the sidewall may be flexible such that the sidewall rolls upon itself with an outer surface of the sidewall at a folding region being folded in a radially inward direction when acted upon by an external force in a direction from the proximal end toward the distal end. The sidewall may be further configured to unroll such that the outer surface of the sidewall at the folding region is unfolded in a radially outward direction when acted upon by the external force in a direction from the distal end toward the proximal end. The syringe assembly further may have a movable closure for selectively enclosing at least a portion of the distal end of the rolling diaphragm syringe within the pressure jacket. The pressure jacket proximal end may have a connection interface for releasably connecting to a fluid injector.

In some embodiments of the present disclosure, a rolling diaphragm syringe may have a piston engagement portion protruding proximally from an end wall. The piston engagement portion may have a central axis and a plurality of grooves recessed inward relative to an outer surface of the piston engagement portion and spaced apart from each other in a direction about the central axis. The piston engagement portion may be configured for engagement with at least one engagement element of a piston of a fluid injector at least during movement of the piston in a proximal direction. At least a portion of the piston engagement portion may be plastically or elastically deformed when engaged with the at least one engagement element of the piston.

In some embodiments of the present disclosure, the plurality of grooves may be spaced apart at equal or unequal intervals along the outer surface of the piston engagement portion in the direction about the central axis. The plurality of grooves may have equal or unequal width measured along the outer surface of the piston engagement portion in the direction about the central axis. The plurality of grooves may have equal or unequal depth measured from the outer surface of the piston engagement portion in a direction toward the central axis. Each groove may have a base and a pair of side surfaces extending from the base to the outer surface of the engagement portion. The plurality of grooves may be continuous over a longitudinal length of the piston engagement portion in a direction along the central axis. At least one of the plurality of grooves may be discontinuous over a longitudinal length of the piston engagement portion in a direction along the central axis.

In some embodiments of the present disclosure, an outer diameter of the piston engagement portion may be uniform or non-uniform in a direction along the central axis. An outer diameter of the piston engagement portion may decrease from a distal end of the piston engagement portion toward a proximal end of the piston engagement portion. In other embodiments, the outer diameter of the piston engagement portion may be greater at the distal and proximal ends along the longitudinal axis than at the middle of the piston engagement portion. In other embodiments, the outer diameter of the piston engagement portion may be greater at the proximal end than along the rest of the longitudinal axis of the piston engagement portion. The piston engagement portion may be monolithically formed with the end wall. The end wall may be concave and has a continuously increasing thickness in a direction toward the longitudinal axis. The piston engagement portion may have a widened portion at a proximal end, and the plurality of grooves may be recessed at least into the widened portion.

Various other aspects of the present disclosure are recited in one or more of the following clauses:

Clause 1. A rolling diaphragm syringe comprising: a proximal end having an end wall; a distal end having an open-ended discharge neck; a sidewall extending between the proximal end and the distal end along a longitudinal axis; a piston engagement portion protruding proximally from a central portion of the end wall, the piston engagement portion having a central axis; and a plurality of grooves recessed inward relative to an outer surface of the piston engagement portion and spaced apart from each other in a direction about the central axis, wherein at least a portion of the sidewall is flexible such that: the sidewall rolls upon itself with an outer surface of the sidewall at a folding region being folded in a radially inward direction when acted upon by an external force in a direction from the proximal end toward the distal end, and the sidewall unrolls with the outer surface of the sidewall at the folding region being unfolded in a radially outward direction when acted upon by the external force in a direction from the distal end toward the proximal end.

Clause 2. The rolling diaphragm syringe according to clause 1, wherein the plurality of grooves are spaced apart at equal or unequal intervals along the outer surface of the piston engagement portion in the direction about the central axis.

Clause 3. The rolling diaphragm syringe according to clause 1 or 2, wherein the plurality of grooves have equal or unequal width measured along the outer surface of the piston engagement portion in the direction about the central axis.

Clause 4. The rolling diaphragm syringe according to any of clauses 1 to 3, wherein the plurality of grooves have equal or unequal depth measured from the outer surface of the piston engagement portion in a direction toward the central axis.

Clause 5. The rolling diaphragm syringe according to any of clauses 1 to 4, wherein each groove has a base and a pair of side surfaces extending from the base to the outer surface of the engagement portion.

Clause 6. The rolling diaphragm syringe according to any of clauses 1 to 5, wherein the plurality of grooves are continuous over a longitudinal length of the piston engagement portion in a direction along the central axis.

Clause 7. The rolling diaphragm syringe according to any of clauses 1 to 5, wherein at least one of the plurality of grooves is discontinuous over a longitudinal length of the piston engagement portion in a direction along the central axis.

Clause 8. The rolling diaphragm syringe according to any of clauses 1 to 7, wherein an outer diameter of the outer surface of the piston engagement portion is uniform in a direction along the central axis.

Clause 9. The rolling diaphragm syringe according to any of clauses 1 to 7, wherein an outer diameter of the outer surface of the piston engagement portion is non-uniform in a direction along the central axis.

Clause 10. The rolling diaphragm syringe according to clause 9, wherein an outer diameter of the outer surface of the piston engagement portion decreases from a distal end of the piston engagement portion toward a proximal end of the piston engagement portion.

Clause 11. The rolling diaphragm syringe according to clause 9, wherein an outer diameter of the outer surface of the piston engagement portion is greater at the distal end and proximal end than in the middle of the piston engagement portion along the central axis.

Clause 12. The rolling diaphragm syringe according to clause 9, wherein an outer diameter of the outer surface of the piston engagement portion is greater at a proximal end of the piston engagement portion than along a remaining portion of the diameter of the piston engagement portion.

Clause 13. The rolling diaphragm syringe according to any of clauses 1 to 12, wherein the piston engagement portion is monolithically formed with the end wall.

Clause 14. The rolling diaphragm syringe according to any of clauses 1 to 13, wherein the piston engagement portion is configured for engagement with at least one engagement element of a piston of a fluid injector at least during movement of the piston in a proximal direction.

Clause 15. The rolling diaphragm syringe according to any of clauses 1 to 14, wherein at least a portion of the piston engagement portion is plastically or elastically deformed when engaged with the at least one engagement element of the piston.

Clause 16. The rolling diaphragm syringe according to any of clauses 1 to 15, wherein the piston engagement portion has a widened portion at a proximal end, and wherein the plurality of grooves are recessed at least into the widened portion.

Clause 17. The rolling diaphragm syringe according to any of clauses 1 to 16, wherein the end wall is concave and has a continuously increasing thickness in a direction toward the longitudinal axis.

Clause 18. The rolling diaphragm syringe according to any of clauses 1 to 17, wherein the central axis of the piston engagement portion is coaxial with the longitudinal axis.

Clause 19. The rolling diaphragm syringe according to any of clauses 1 to 18, wherein the rolling diaphragm syringe is in a first state where the end wall is inverted and rolled inwardly toward the distal end such that an internal volume of the rolling diaphragm syringe is empty of fluid.

Clause 20. A syringe assembly for a fluid delivery system, the syringe assembly comprising: a pressure jacket having a pressure jacket distal end, a pressure jacket proximal end, and a throughbore extending between the pressure jacket distal end and the pressure jacket proximal end along a longitudinal axis; and a rolling diaphragm syringe disposed within the throughbore of the pressure jacket, the rolling diaphragm syringe comprising: a proximal end having an end wall; a distal end having an open-ended discharge neck; a sidewall extending between the proximal end and the distal end along a longitudinal axis; a piston engagement portion protruding proximally from a central portion of the end wall, the piston engagement portion having a central axis; and a plurality of grooves recessed inward relative to an outer surface of the piston engagement portion and spaced apart from each other in a direction about the central axis, wherein at least a portion of the sidewall is flexible such that: the sidewall rolls upon itself with an outer surface of the sidewall at a folding region being folded in a radially inward direction when acted upon by an external force in a direction from the proximal end toward the distal end, and the sidewall unrolls with the outer surface of the sidewall at the folding region being unfolded in a radially outward direction when acted upon by the external force in a direction from the distal end toward the proximal end.

Clause 21. The syringe assembly according to clause 21, further comprising a movable closure for selectively enclosing at least a portion of the distal end of the rolling diaphragm syringe within the pressure jacket.

Clause 22. The syringe assembly according to clause 21 or clause 22, wherein the pressure jacket proximal end has a connection interface for releasably connecting to a fluid injector.

Clause 23. A rolling diaphragm syringe comprising: a piston engagement portion protruding proximally from an end wall, the piston engagement portion having a central axis; and a plurality of grooves recessed inward relative to an outer surface of the piston engagement portion and spaced apart from each other in a direction about the central axis, wherein the piston engagement portion is configured for engagement with at least one engagement element of a piston of a fluid injector at least during movement of the piston in a proximal direction, and wherein at least a portion of the piston engagement portion is plastically or elastically deformed when engaged with the at least one engagement element of the piston.

Clause 24. The rolling diaphragm syringe according to clause 24, wherein the plurality of grooves are spaced apart at equal or unequal intervals along the outer surface of the piston engagement portion in the direction about the central axis.

Clause 25. The rolling diaphragm syringe according to clause 23 or 24, wherein the plurality of grooves have equal or unequal width measured along the outer surface of the piston engagement portion in the direction about the central axis.

Clause 26. The rolling diaphragm syringe according to any of clauses 23 to 25, wherein the plurality of grooves have equal or unequal depth measured from the outer surface of the piston engagement portion in a direction toward the central axis.

Clause 27. The rolling diaphragm syringe according to any of clauses 23 to 26, wherein each groove has a base and a pair of side surfaces extending from the base to the outer surface of the engagement portion.

Clause 28. The rolling diaphragm syringe according to any of clauses 23 to 27, wherein the plurality of grooves are continuous over a longitudinal length of the piston engagement portion in a direction along the central axis.

Clause 29. The rolling diaphragm syringe according to any of clauses 23 to 27, wherein at least one of the plurality of grooves is discontinuous over a longitudinal length of the piston engagement portion in a direction along the central axis.

Clause 30. The rolling diaphragm syringe according to any of clauses 23 to 29, wherein an outer diameter of the piston engagement portion is uniform in a direction along the central axis.

Clause 31. The rolling diaphragm syringe according to any of clauses 23 to 29, wherein an outer diameter of the piston engagement portion is non-uniform in a direction along the central axis.

Clause 32. The rolling diaphragm syringe according to clause 31, wherein an outer diameter of the piston engagement portion decreases from a distal end of the piston engagement portion toward a proximal end of the piston engagement portion.

Clause 33. The rolling diaphragm syringe according to clause 31, wherein an outer diameter of the outer surface of the piston engagement portion is greater at the distal end and proximal end than in the middle of the piston engagement portion along the central axis.

Clause 34. The rolling diaphragm syringe according to clause 31, wherein an outer diameter of the outer surface of the piston engagement portion is greater at a proximal end of the piston engagement portion than along a remaining portion of the diameter of the piston engagement portion.

Clause 35. The rolling diaphragm syringe according to any of clauses 23 to 34, wherein the piston engagement portion is monolithically formed with the end wall.

Clause 36. The rolling diaphragm syringe according to any of clauses 23 to 35, wherein the end wall is concave and has a continuously increasing thickness in a direction toward the longitudinal axis.

Clause 37. The rolling diaphragm syringe according to any of clauses 23 to 36, wherein the piston engagement portion has a widened portion at a proximal end, and the plurality of grooves are recessed at least into the widened portion.

Clause 38. A method for forming a rolling diaphragm syringe, the method comprising molding a plurality of inwardly recessed grooves onto at least a portion of an outer surface of an engagement portion that protrudes proximally from a proximal end wall of the rolling diaphragm syringe.

Clause 39. The method of clause 38, wherein the plurality of inwardly recessed grooves are molded into at least the portion of the outer surface of the engagement portion during an injection molding process of a preform that is blow-molded to form the rolling diaphragm syringe.

Clause 40. The method of clause 38, wherein the plurality of inwardly recessed grooves are molded into at least the portion of the outer surface of the engagement portion during a blow-molding process to form the rolling diaphragm syringe.

Clause 41. A method for engaging an engagement portion of a rolling diaphragm syringe, the method comprising: engaging an edge surfaces of a terminal end of one or more engagement elements of a piston with an outer surface of the engagement portion, wherein at least a portion of the outer surface has a plurality of inwardly recessed grooves thereon; and at least partially embedding the edge surfaces of the terminal end of the one or more engagement elements into a material of the plurality of inwardly recessed grooves on at least the portion of the outer surface of the engagement portion.

Clause 42. The method of claim 41, further comprising retracting the piston in a proximal direction, wherein retracting the piston initiates engaging the edge surfaces of the terminal end of the one or more engagement elements of the piston with an outer surface of the engagement portion.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is a front perspective view of a fluid injector in accordance with some embodiments of the present disclosure;

FIG. 2A is a side cross-sectional view of a rolling diaphragm syringe in accordance with some embodiments of the present disclosure, with the rolling diaphragm syringe shown in an unrolled state;

FIG. 2B is a side cross-sectional view of the rolling diaphragm syringe of FIG. 2A with the rolling diaphragm syringe shown in a rolled state;

FIG. 3A is a side cross-sectional view of a rolling diaphragm syringe and a syringe engagement mechanism of a fluid injector shown in an open state or configuration;

FIG. 3B is a side cross-sectional view of the rolling diaphragm syringe and the syringe engagement mechanism of the fluid injector of FIG. 3A, with the syringe engagement mechanism shown in a closed state or configuration;

FIG. 4A is a bottom perspective view of a rolling diaphragm syringe having a grooved engagement portion in accordance with some embodiments of the present disclosure;

FIG. 4B is a bottom perspective view of a rolling diaphragm syringe having a grooved engagement portion in accordance with some embodiments of the present disclosure;

FIG. 4C is a bottom perspective view of a rolling diaphragm syringe having a grooved engagement portion in accordance with some embodiments of the present disclosure;

FIG. 5A is a partial perspective cross-sectional view of a rolling diaphragm syringe having a grooved engagement portion and a syringe engagement mechanism of a fluid injector in accordance with some embodiments of the present disclosure, with the syringe engagement mechanism shown in a closed position;

FIG. 5B is a side cross-sectional view of the rolling diaphragm syringe having a grooved engagement portion and the syringe engagement mechanism shown in FIG. 5A;

FIG. 5C is a detailed perspective view of the grooved engagement portion of the rolling diaphragm syringe shown in FIG. 5A;

FIG. 5D is a detailed cross-sectional view of the grooved engagement portion of the rolling diaphragm syringe shown in FIG. 5C;

FIG. 5E is a detailed side cross-sectional view of a pointed terminal end or edge of the syringe engagement mechanism during engagement with the engagement portion of the rolling diaphragm syringe shown in FIG. 5C;

FIG. 6A is a side cross-sectional view of an engagement portion of the a rolling diaphragm syringe and a syringe engagement mechanism in accordance with some embodiments of the present disclosure; and

FIG. 6B is a side cross-sectional view of an engagement portion of the a rolling diaphragm syringe and a syringe engagement mechanism in accordance with some embodiments of the present disclosure.

### DETAILED DESCRIPTION OF THE DISCLOSURE

The figures and specification generally show non-limiting embodiments of the systems and methods of the present disclosure. While the description presents various embodiments of the devices, it should not be interpreted in any way as limiting the disclosure. Furthermore, modifications, concepts, and applications of the disclosure's embodiments are to be interpreted by those skilled in the art as being encompassed, but not limited to, the illustrations and descriptions herein.

The following description is provided to enable those skilled in the art to make and use the described embodiments contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures.

When used in relation to a rolling diaphragm syringe, the term “proximal” refers to a portion of a rolling diaphragm syringe nearest to a fluid injector when a rolling diaphragm syringe and/or a pressure jacket is oriented for connecting to a fluid injector.

When used in relation to a rolling diaphragm syringe, the term “distal” refers to a portion of a rolling diaphragm syringe farthest away from a fluid injector when a rolling diaphragm syringe and/or a pressure jacket is oriented for connecting to a fluid injector.

When used in relation to a rolling diaphragm syringe, the term “radial” refers to a direction in a cross-sectional plane normal to a longitudinal axis of a rolling diaphragm syringe extending between proximal and distal ends.

When used in relation to a rolling diaphragm syringe, the term “circumferential” refers to a direction around an inner or outer surface of a sidewall of a rolling diaphragm syringe.

When used in relation to a rolling diaphragm syringe, the term “axial” refers to a direction along a longitudinal axis of a rolling diaphragm syringe extending between the proximal and distal ends.

The term “flexible”, when used in connection with a rolling diaphragm syringe, means that at least a portion of a rolling diaphragm syringe, such as a sidewall of a rolling diaphragm syringe, is capable of bending or being bent to change a direction in which it extends.

The terms “roll over”, “rolling over”, and “rolls upon itself”, when used in connection with a rolling diaphragm syringe, refer to an ability of a first portion of a rolling diaphragm syringe, such as a proximal portion of a sidewall of a rolling diaphragm syringe, to bend approximately 180° relative to a second portion of a rolling diaphragm syringe, such as a distal portion of a sidewall of a rolling diaphragm syringe, when urged by a piston of a fluid injector or a rolling fixture. In a similar manner, the term “unrolls”, when used in connection with a rolling diaphragm syringe, refers to the ability of the first portion of the sidewall of the rolling diaphragm syringe to unroll in an opposite direction relative to the second portion of the sidewall of the rolling diaphragm syringe.

It is to be understood, however, that the disclosure may assume alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the examples disclosed herein are not to be considered as limiting.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

With reference to FIG. 1, a fluid injector 10 according to certain embodiments may include at least one injector head 12 and an injector housing 14. The fluid injector 10 may be supported on a support structure 13. In some embodiments, such as shown in FIG. 1, the fluid injector 10 may include two injector heads 12 arranged in a side-by-side or any other orientation. Each injector head 12 may be formed at a front end of the injector housing 14 and may be configured for receiving and retaining at least one pressure jacket 16. While FIG. 1 illustrates the fluid injector 10 with two injector heads 12, each with a corresponding pressure jacket 16, other examples of the fluid injector 10 may include a single injector head 12 and a corresponding pressure jacket 16 or more than two injector heads 12 with a corresponding number of pressure jackets 16. In other embodiments, the pressure jacket 16 may be incorporated into the injector head 12 (i.e., located within the injector head).

With continued reference to FIG. 1, each pressure jacket 16 has a proximal end 37, a distal end 39, and a throughbore 41 extending between the proximal end 37 and the distal end 39. Each pressure jacket 16 has a removable closure 45 connectable to the distal end 39 for enclosing at least a portion of the distal end of a rolling diaphragm syringe, as described herein. The proximal end 37 has a connection interface 43 for releasably connecting to a fluid injector 10.

Each injector head 12 includes at least one piston 19 (shown in FIGS. 3A-3B), such as a reciprocally driven piston moved by a motor (not shown) which is operated by a controller (not shown). Each piston 19 may be configured to extend into and from the respective injector head 12 through an opening in the distal end of the injector housing 14. The pistons 19 impart a motive force to at least a portion of rolling diaphragm syringes 20 disposed in respective pressure jackets 16, as described herein.

With continued reference to FIG. 1, the fluid injector 10 is configured to receive a rolling diaphragm syringe 20 within each pressure jacket 16. The pressure jacket 16 is typically a reusable component, while the rolling diaphragm syringe 20 may be a single-use component which is disposed of after an injection procedure. In some embodiments, however, the rolling diaphragm syringe 20 may be a multi-use component.

The fluid injector 10 may have at least one bulk fluid source 21 for filling the rolling diaphragm syringes 20 with fluid F. The fluid F may be imaging contrast agent, saline, or any other medical fluid. At least one fluid path set 23 may be fluidly connected with a discharge end of each rolling diaphragm syringe 20 for delivering fluid from the rolling diaphragm syringe 20 through tubing connected to a catheter, needle, or other fluid delivery connection (not shown) inserted into a patient at a vascular access site.

Fluid delivery or filling may be regulated by a controller 29. The controller 29 may operate various pistons, valves, and/or flow regulating structures of the fluid injector 10 to regulate the flow of fluid from the rolling diaphragm syringes 20 to the patient based on user selected injection parameters, such as injection flow rate, duration, total injection volume, and/or ratio of contrast media and saline. The controller 29 may be further configured to control filling of the rolling diaphragm syringes 20 with fluid. Examples of suitable front-loading fluid injectors that may be used or modified for use with the herein-described system, including at least one pressure jacket 16 and rolling diaphragm syringe 20, are disclosed in International Application Publication No. WO 2015/164783 and International Application Publication No. WO 2016/172467, the disclosures of which are incorporated herein by reference. The controller 29 may be hard-wired or wirelessly connected to a processor (not shown) configured for providing instructions to the controller and allowing programming of injection parameters, as well as storing and communicating information on injection procedures to a technician or hospital information network.

With reference to FIGS. 2A-2B, in certain examples, the rolling diaphragm syringe 20 generally includes a hollow body 25 defining an interior volume 27. The body 25 has a forward or distal end 28, a rearward or proximal end 30, and a flexible sidewall 32 extending therebetween along a longitudinal axis 31. In some embodiments, the rolling diaphragm syringe 20 may be pre-filled with a medical fluid. In other embodiments, the rolling diaphragm syringe 20 may be initially empty of fluid and optionally provided in a rolled configuration.

The sidewall 32 of the rolling diaphragm syringe 20 defines a soft, pliable or flexible, yet self-supporting body that is configured to roll upon itself under the action of the piston 19. In particular, the sidewall 32 is configured to roll such that its outer surface 33 is folded and inverted in a radially inward direction at a folding region 35 as the piston 19 (shown in FIGS. 3A-3B) is moved in a distal direction and unrolled and unfolded in the opposite manner in a radially outward direction as the piston 19 is retracted in a proximal direction. The sidewall 32 may have a smooth, substantially uniform structure, or it may have one or more ribs provided thereon to facilitate the rollover during an injection procedure. In some embodiments, the sidewall 32 may have a textured surface, or a combination of a smooth surface and a textured surface. One or more indicia (not shown) may be formed on the sidewall 32. In some embodiments, the sidewall 32 may have a uniform thickness along its longitudinal length. In other embodiments, the sidewall 32 may have a non-uniform thickness along its longitudinal length. In specific embodiments, the sidewall 32 at or near the distal end 28 may be substantially rigid.

With continued reference to FIGS. 2A-2B, the rearward or proximal portion 30 of the sidewall 32 has a closed end wall 34, and a forward or distal portion 28 of the sidewall 32 defines an open-ended discharge neck 36 opposite the closed end wall 34. The closed end wall 34 may have a concave shape to facilitate the initiation of the inversion or rolling of the sidewall 32 and/or to provide a receiving pocket to receive a distal end of the piston 19. For example, the closed end wall 34 may define a receiving end pocket 38 for interfacing directly with a similarly-shaped piston 19. In particular examples, at least a portion of the piston 19 may be shaped to substantially match the shape of the closed end wall 34 or, alternatively, pressure from the piston 19 as it is moved distally may conform the end wall 34 to substantially match the shape of at least a portion of the piston 19. The closed end wall 34 may have a non-uniform thickness. In some embodiments, the thickness of the end wall 34 may increase in a direction extending toward the longitudinal axis 31 of the rolling diaphragm syringe 20. In certain embodiments, at least a portion of the end wall 34 may be thicker near the center and thinner near the connection with the sidewall 32.

With continued reference to FIGS. 2A-2B, the body 25 of the rolling diaphragm syringe 20 is adapted to be removably received in the interior portion of the throughbore 41 of the pressure jacket 16. The distal end 28 of the rolling diaphragm syringe 20 may be removably attachable to the pressure jacket 16, or configured with a retention surface to interact with a retaining force to retain the rolling diaphragm syringe 20 within the pressure jacket 16. The distal end 28 may have a frusto-conical shape that gradually narrows from the sidewall 32 to the discharge neck 36. In certain embodiments, the discharge neck 36 may terminate in a discharge port 40 having a connector 42 for connecting to a cap, fluid path set, and/or other connection element. In some embodiments, the connector 42 is a threaded interface having one or more threads. In other examples, the connector 42 may have a luer-type connection. In further examples, the sidewall 32 may have one or more lips or grooves that interact with corresponding grooves or lips on the pressure jacket 16 to releasably retain the rolling diaphragm syringe 20 within the pressure jacket 16 during an injection procedure.

The outer diameter of the rolling diaphragm syringe 20 may be dimensioned such that the rolling diaphragm syringe 20 fits within the interior space defined by the inner surface of the throughbore 41 of the pressure jacket 16. In some embodiments, the rolling diaphragm syringe 20 fits snuggly but removably within the pressure jacket 16 such that the outer surface 33 of the rolling diaphragm syringe 20 abuts at least a portion of the inner surface of the walls of the pressure jacket 16. In other embodiments, the rolling diaphragm syringe 20 fits loosely within the pressure jacket 16 such that there is a gap between at least a portion of the outer surface 33 of the rolling diaphragm syringe 20 and the inner surface of the pressure jacket 16. The rolling diaphragm syringe 20 may be expanded under pressure during a pressurized injection procedure such that the outer surface 33 of the rolling diaphragm syringe 20 abuts the inner surface of the pressure jacket 16. Examples of suitable pressure jacket features are described in International Application Publication No. WO 2018/053074, the entire disclosure of which is incorporated herein by reference.

The end wall 34 may have a central portion 44 having a substantially dome-shaped structure and a piston engagement portion 46 (hereinafter referred to as “engagement portion 46”) extending proximally from the central portion 44. The engagement portion 46 has a central axis 47 that extends along a longitudinal length of the engagement portion 46. The central axis 47 may be coaxial with the longitudinal axis 31 of the rolling diaphragm syringe 20. In some embodiments, the engagement portion 46 may extend in a proximal direction from an approximate midpoint of the central portion 44.

In some embodiments, an outer diameter Dₒ of the engagement portion 46 may be uniform, such that the engagement portion 46 has a substantially cylindrical structure throughout its longitudinal length. In other embodiments, the diameter Dₒ of the engagement portion 46 may be non-uniform. For example, the diameter Dₒ of the engagement portion 46 may gradually decrease or increase in the proximal direction along the central axis 47. While the engagement portion 46 is shown in FIGS. 2A-2B as being substantially flush with the proximal end 30 of the rolling diaphragm syringe 20, in certain examples the engagement portion 46 may extend proximally beyond the proximal end 30 of the rolling diaphragm syringe 20.

The engagement portion 46 may be monolithically formed with the syringe body 25, such as with the end wall 34. For example, the engagement portion 46 may be formed on an injection molded preform, which may be blow-molded to form the syringe body 25. In some embodiments, the engagement portion 46 may be removably or non-removably attached to the central portion 44 of the end wall 34, such as by welding, adhesion, or clip attachment, or other fastening mechanism. The engagement portion 46 is configured for interacting with a syringe engagement mechanism 48 (hereinafter referred to as “engagement mechanism 48”) on the piston 19 of the fluid injector 10, as described herein.

The rolling diaphragm syringe 20 may be made of any suitable medical-grade plastic or polymeric material and in various embodiments may be a clear or substantially translucent plastic material. The material of the rolling diaphragm syringe 20 may be selected to meet the required tensile and planar stress requirements, water vapor transmission requirements, and/or chemical/biological compatibility requirements.

In certain examples, suitable rolling diaphragm syringes 20 include a rolling diaphragm-type syringe as described in WO 2015/164783 and WO 2016/172467 having a flexible thin sidewall 32 which rolls upon itself when acted upon by the piston 19 such that an outer surface of the sidewall 32 at a folding region 35 is folded in a radially inward direction as the piston 19 is advanced from the proximal end 30 to the distal end 28 and such that the outer surface of the sidewall 32 at the folding region 35 is unfolded in a radially outward direction as the piston 19 is retracted from the distal end 28 toward the proximal end 30. Upon pressurization of the rolling diaphragm syringe 20 by distal movement of the piston 19, the fluid pressure within the rolling diaphragm syringe causes the sidewall 32 to expand radially outward. This effect is enhanced by the relative thinness of the syringe sidewall 32 compared to conventional syringes. As the syringe sidewall 32 expands radially outward, it contacts the interior surface of the pressure jacket 16, which limits further expansion of the syringe sidewall 32.

FIGS. 3A-3B show the rolling diaphragm syringe 20 in combination with one embodiment of a engagement mechanism 48 of the piston 19 in accordance with some embodiments of the present disclosure. The components of the rolling diaphragm syringe 20 shown in FIGS. 3A-3B are substantially similar to the components of the rolling diaphragm syringe 20 described herein with reference to FIGS. 2A-2B. The engagement portion 46 of the rolling diaphragm syringe 20 is configured for interacting with one or more engagement pins or surfaces 86 of the engagement mechanism 48 that reversibly move radially inward and outward to engage and disengage, respectively, the engagement portion 46 of the rolling diaphragm syringe 20. In various examples, the inward/outward movement of engagement elements or surfaces 56 may be effected by a proximal/distal movement of the piston 19 or may be moved inward/outward by one or more motive forces provided by the fluid injector 10. The engagement elements or surfaces 56 may be moved radially inward/outward via linear movement, arcuate movement, or a combination of linear and arcuate movement.

In various aspects, movement of the piston 19 in the proximal direction may cause the engagement elements or surfaces 56 to move inwards and contact the engagement portion 46 of the syringe 30 such that the end wall 34 of the rolling diaphragm syringe 20 may be moved proximally with the proximal movement of the piston 19. Conversely, movement of the piston 19 in a distal direction may cause the engagement elements or surfaces 56 to be released from contacting the engagement portion 46 of the rolling diaphragm syringe 20 such that the rolling diaphragm syringe 20 may be removed from the pressure jacket 16 and the injector 10. In various examples, the piston 19 may be movable by a motor drive, a solenoid drive, or it may be an electro-active polymer. Syringe engagement mechanism 48 may be any of the syringe engagement mechanisms described in International Application Publication No. WO 2018/075386, the disclosure of which is incorporated herein by reference.

With continued reference to FIGS. 3A-3B, according to certain embodiments the piston 19 may have an outer piston sleeve 50 and an abutment section 52 movably received within the outer piston sleeve 50 at a distal end thereof. The outer piston sleeve 50 has a substantially cylindrical structure with an open proximal end and an open distal end. The abutment section 52 has an outer engagement surface 57 at its distal end for engaging at least a portion of the proximal end 30 of the rolling diaphragm syringe 20 when the piston 19 is advanced distally to engage with the rolling diaphragm syringe 20. In some embodiments, the abutment section 52 of the piston 19 may contact at least a portion of the proximal end 30 of the rolling diaphragm syringe 20, such as the end wall 34. The outer engagement surface 57 may be shaped to correspond to the shape of the end wall 34 such that the outer engagement surface 57 is in surface-to-surface contact with at least a portion of the end wall 34 when engaged with the rolling diaphragm syringe 20. The outer engagement surface 57 and outer piston sleeve 50 define a surface over which the sidewall 32 of the rolling diaphragm syringe 20 may roll over during a fluid filling or a fluid delivery process due to proximal or distal movement of the piston 19, respectively.

An opening 55 is formed in a central portion of the abutment section 52. The opening 55 is configured to receive at least a portion of the engagement portion 46 of the rolling diaphragm syringe 20 when the abutment section 52 substantially contacts the end wall 34 of the syringe 20. In certain embodiments, an inner diameter of the opening 55 is larger than an outer diameter of the widest portion of the engagement portion 46 to allow free insertion of the engagement portion 46 into the opening 55 during distal movement of the piston 19 toward the end wall 34 of the rolling diaphragm syringe 20 or proximal movement of the end wall 34 of the rolling diaphragm syringe 20 toward the piston 19, for example during insertion of the rolling diaphragm syringe 20 into the pressure jacket 16, as well as free removal of the engagement portion 46 from the opening 55 during removal of the rolling diaphragm syringe 20.

In some embodiments, the abutment section 52 may be axially movable relative to the outer piston sleeve 50, which is held in a substantially fixed position, for example due to friction between the outer piston sleeve 50 and the piston 19. The abutment section 52 is movable or slidable in an axial direction relative to the outer piston sleeve 50 to control the state or position of one or more engagement elements 56, as described herein. The movement of the abutment section 52 relative to the outer piston sleeve 50 is configured to allow engagement or disengagement of the one or more engagement elements 56 with the engagement portion 46 of the rolling diaphragm syringe 20, as described herein.

With continued reference to FIGS. 3A-3B, the syringe engagement mechanism 48 includes one or more engagement elements 56 configured for contacting the engagement portion 46 of the rolling diaphragm syringe 20 during at least a proximal movement of the piston 19 to facilitate unrolling of the rolling diaphragm syringe 20. The one or more engagement elements 56 and optionally a plurality of engagement elements 56 are spaced apart radially relative to the engagement portion 46 of the rolling diaphragm syringe 20 when the rolling diaphragm syringe 20 is inserted into the injector 10. In some embodiments, a single engagement element 56 may be configured to contact the engagement portion 46 of the rolling diaphragm syringe 20. In other embodiments, at least one pair of engagement elements 56 may be positioned opposite one another with the engagement portion 46 of the rolling diaphragm syringe 20 disposed therebetween. The engagement elements 56 may be spaced apart at equal or unequal angular intervals from one another.

The engagement elements 56 may be movable between a first position (FIG. 3A), where the engagement elements 56 do not contact the engagement portion 46 of the rolling diaphragm syringe 20, and a second position (FIG. 3B), wherein the engagement elements 56 contact the outer surface of the engagement portion 46 of the rolling diaphragm syringe 20. The engagement elements 56 may be movable in a radially inward/outward direction linearly or non-linearly, such as in an arcuate movement, for example due to pivoting about a pivot point 83. In some embodiments, the engagement elements 56 may have a sharpened edge or pointed terminal ends 86 for embedding at least a portion of the engagement elements 56 into the material of the engagement portion 46 of the rolling diaphragm syringe 20 when the engagement elements 56 are positioned in the second position (FIG. 3B). In the second position, the edge of the terminal ends 86 of the engagement elements 56 may dig into and become at least partially embedded into the material of the engagement portion 46.

According to various examples, the inward/outward movement of the engagement elements 56 in FIGS. 3A-3B and depth of engagement of the engagement element 56 and the engagement portion 46 may at least in part be dependent on proximal/distal movement of the piston 19. For example, the engagement element may be angled so that the sharpened edge or pointed end are directed at a proximal angle such that during initial movement of the piston 19 in the proximal direction, the engagement elements 56 may be advanced radially inward to an initial contact position where the engagement elements 56 contact the outer surface of the engagement portion 46 of the rolling diaphragm syringe 20. With continued proximal movement of the piston 19, the angled edge or point of the engagement elements 56 may continue to move in a radially inward direction from the initial contact position such that the engagement elements 56 and pointed edge/terminal ends 86 dig further into or become embedded within the material of the engagement portion 46 of the rolling diaphragm syringe 20 by the proximal movement of the piston 19 to increase the holding force on the engagement portion 46 and therefore the end wall 34 of the rolling diaphragm syringe 20. The engagement elements 56 may move to a final contact position having a maximum radial displacement from the initial contact position at a final proximal position of the piston 19. In some embodiments, radial movement of the engagement elements 56 may further be a function of proximal movement of the piston 19 in a direction of arrow B shown in FIG. 3B. That is, as the piston 19 is moved further in the proximal direction, the force of the “bite” or radially inward force between the engagement elements 56 and the edge/pointed terminal ends 86 with the engagement portion 46 increases so that any proximal slipping of the pointed terminal ends 86 through the material of the engagement portion 46 is counteracted. Further, as the edge/pointed terminal ends 86 dig into the material of the engagement portion 46 a portion of the material of the engagement portion 46 may be forced radially outward forming a berm 98 of surface material proximal to the edge/pointed terminal ends 86 of the engagement elements 56 that increases the holding force of the engagement elements 56 with the engagement portion 46. According to the various embodiments described herein, the plurality of grooves 94 in the surface of the engagement portion 46 may increase the depth that the engagement elements 56 embeds into the engagement portion 46 and/or the height of berm 98, thereby increasing the “bite” and retaining force between the engagement elements 56 and the engagement portion 46.

In other embodiments, the inward/outward movement of the engagement elements 56 may occur independently of the proximal/distal movement of the piston 19 due to operation of a drive mechanism 88. The drive mechanism 88 may be configured for controlling the movement of the engagement elements 56 between a first, disengaged position (FIG. 3A) and a second, engaged position (FIG. 3B). The drive mechanism 88 may be associated with at least a portion of the piston 19. In various examples, the drive mechanism 88 may be mechanically, electrically, pneumatically, and/or hydraulically operated. For example, the drive mechanism 88 may have an electric or electromechanical mechanism, such as a linear or rotary electric motor, or a solenoid. In other examples, the drive mechanism 88 may be activated/deactivated by an electromagnetic mechanism, an electroactive polymer mechanism, or a shape-memory alloy-based mechanism (such as nitinol wire). Various combinations of these mechanisms are also contemplated at being within the scope of the present disclosure. In some embodiments, the drive mechanism 88 may be selectively energized, such as during proximal or distal movement of the piston 19. In other examples, the drive mechanism 88 may be constantly energized, regardless of whether the piston 19 is stationary, or moving in the proximal or distal direction.

With reference to FIGS. 4A-4C, rolling diaphragm syringes 20 with various examples of the engagement portion 46 are shown. In each example shown in FIGS. 4A-4C, the engagement portion 46 is configured for interacting with an engagement mechanism 48 on the piston 19 of the fluid injector 10, as described herein. Each engagement portion 46 extends in the proximal direction along the central axis 47 from the central portion 44 of the end wall 34. The engagement portion 46 is monolithically formed with the rolling diaphragm syringe 20 such that a base 90 of the engagement portion 46 is integrally formed with the end wall 34 of the syringe 19. In some embodiments, the base 90 of the engagement portion 46 may be non-removably attached to the central portion 44 of the end wall 34, such as by welding, adhesion, or clip attachment, or other fastening mechanism.

With continued reference to FIGS. 4A-4C, the engagement portion 46 has a body 92 protruding proximally from the base 90. In some embodiments, such as shown in FIG. 4C, an outer diameter D_{O} of the body 92 may be substantially uniform throughout a longitudinal length of the body 92. In other embodiments, such as shown in FIGS. 4A-4B, the outer diameter D_{O} of the body 92 may be non-uniform. For example, the diameter D_{O} of the body 92 may decrease in a proximal direction extending away from the base 90. In other embodiments, the outer diameter D_{O} of the body 92 may increase in the proximal direction extending away from the base 90. In further examples, the diameter D_{O} of the body 92 may increase over a first portion of the longitudinal length of the body 92 in a proximal direction extending away from the base 90 and decrease over a second portion of the longitudinal length of the body 92, or vice versa. Changes in the outer diameter D_{O} of the engagement portion 46 over the longitudinal length of the body 92 may have an impact of the holding force between the engagement elements 56 of the piston 19 and the engagement portion 46 of the rolling diaphragm syringe 20.

With continued reference to FIGS. 4A-4C, the body 92 of the engagement portion 46 has one or more grooves 94 recessed radially inward relative to an outer surface 96 of the body 92. In this manner, the body 96 has a core 97 with one or more splines 99 extending radially outward and defined between adjacent grooves 94. The one or more grooves 94 may be formed during manufacture of the rolling diaphragm syringe 20, such as during the molding operation to form the rolling diaphragm syringe 20. For example, in one embodiment, the one or more grooves 94 may be formed during an injection molding operation to form a preform that may be ultimately blow-molded to form the rolling diaphragm syringe 20. In another embodiment, the one or more grooves 94 may be formed during the blow-molding process to form the rolling diaphragm syringe 20 where, as the engagement portion 46 on the preform moves into its final position in the blow-mold, the material of the engagement portion 46 is heated to the glass transition temperature and the one or more grooves 94 are press molded into the body 92 of the engagement portion 46. In some embodiments, the one or more grooves 94 may be formed on the body 92 in a separate manufacturing operation after the rolling diaphragm syringe 20 is formed. For example, the one or more grooves 94 may be cut into the outer surface 96 of the body 92 after the rolling diaphragm syringe 20 is molded. In another embodiment, the one or more grooves 94 may be molded into the engagement portion 46 during a rolling process, as described in PCT International Application No. PCT/US2019/018404, the disclosure of which is incorporated by reference, where before, during, or after the rolling process, the material of the engagement portion 46 is heated to the glass transition temperature and the one or more grooves 94 are press molded into the body 92 of the engagement portion 46. In certain embodiments, the one or more grooves 94 may be identical to each other. In some embodiments, at least one of the one or more grooves 94 may be different from the other grooves 94. While FIGS. 4A-4C show the one or more grooves 94 as having a substantially through-shaped form with a base and a pair of sides extending from the base, other examples of the one or more grooves 94 may have other geometric shapes, for example a rounded or curved base surface.

The one or more grooves 94 have a length L along at least a portion of the longitudinal length of the body 92 in a direction along the central axis 47. In some embodiments, the length L may be continuous over at least a portion of the longitudinal length of the body 92 in the direction along the central axis 47. In other embodiments, the one or more grooves 94 may be discontinuous along the longitudinal length of the body 92 in the direction along the central axis 47. The one or more grooves 94 may be spaced apart at equal or unequal intervals along the outer surface 96 of the body 92 of the engagement portion 46 in a direction about the central axis 47. The one or more grooves 94 may have equal or unequal widths W measured along the outer surface 96 of the engagement portion 46 in the direction about the central axis 47. While FIGS. 4A-4C show a plurality of grooves 94 having equal widths W and/or equal circumferential spacing therebetween, other examples of the engagement portion 46 may have grooves with unequal widths W and/or unequal circumferential spacing. While FIGS. 4A-4C show a plurality of grooves 94 having extending parallel to the central axis 47, in other embodiments, the one or more grooves may be formed around the circumference of the body 92. According to another embodiment, the one or more grooves may be formed in a spiral arrangement around the body 92 along the length of the body. Other embodiments may include combinations of these groove configurations, such as a cross-hatched pattern over the length of the body 92. In still other embodiments, a first portion of the body 92 may include one or more circumferential grooves a second portion may include one or more longitudinal grooves. Further, in various embodiments, the grooves may be along a portion of the length of the body 92, such that a portion of the engagement portion 46 does not have any surface grooves.

With continued reference to FIGS. 4A-4C, the one or more grooves 94 have a depth D in a radially inward direction from the outer surface 96 of the body 92. In some embodiments, the depth D of the one or more grooves may be in the range of 0.003 to 0.080 inches, such as 0.020 to 0.080 inches. The depth D may be uniform along the entire length L of each of the one or more grooves 94, such as shown in FIGS. 4A-4B. In some embodiments, the depth D may be non-uniform along at least a portion of the length L of at least one of the one or more grooves 94. For example, with reference to FIG. 4C, the depth D may increase along the length L of the one or more grooves 94 in the proximal direction of the body 92 extending from the base 90. In such examples, the core 97 may be tapered, such that its diameter increases or decreases in a proximal direction of the body 92, while an outer diameter of the body 92 may be uniform. Alternatively, the core 97 may have a uniform diameter, while an outer diameter of the body 92 may increase or decrease in a proximal direction of the body 92.

In some embodiments, one or more radially spaced apart grooves 94 may be combined with one or more axially spaced grooves such that the engagement portion 46 of the rolling diaphragm syringe 20 has a plurality of “dots” defined by the radial and axial grooves. For example, the one or more radial grooves may be similar to any of the grooves 94 described herein with reference to FIGS. 4A-4C while the one or more axial grooves may be oriented at an angle relative to the radial grooves, such as substantially perpendicular to the radial grooves, to define a pattern of “dots” or other shapes on the outer surface 96 of the body 92. In some embodiments, the “dots” may have a rectangular shape, a circular shape, or any other geometric shape.

With reference to FIG. 6A, the body 92 of the engagement portion 46 may have a first diameter D1 at its distal end that is connected to the end wall 34 of the rolling diaphragm syringe 20. The diameter of the body 92 may decrease from the first diameter D1 at the distal end to a second diameter D2 at an intermediate portion 63 of the body 92 and then increases from the second diameter D2 to a third diameter D3 at the proximal end of the body 92. In this manner, the body 92 may have a narrowed diameter at the intermediate portion 63 between the proximal and distal ends. The first diameter D1 and the third diameter D3 are both larger than the second diameter D3. The first diameter D1 and the third diameter D3 may be equal to each other, or one of the two may be larger than the other.

With reference to FIG. 6B, body 92 of the engagement portion 46 may have a widened portion 65 at a proximal end 67. The widened portion 65 may extend radially outward relative to an outer surface of the body 92 such that a diameter of the widened portion 65 is larger than a diameter of the body 92 distal of the widened portion 65. In some embodiments, the one or more grooves 94 (shown in FIGS. 4A-4C) may be provided on at least one of the body 92 and the widened portion 65. In the embodiments illustrated in FIGS. 6A and 6B, the increased diameter of the proximal end of the body 92 may allow better gripping or “bite” between the engagement elements 56 and the engagement portion 46.

Without intending to be bound by any theory, it has been found that the one or more grooves 94 contributes to a more consistent physical contact force between the engagement portion 46 of the rolling diaphragm syringe 20 and the engagement elements 56 of the piston 19 during proximal movement of the piston 19. With reference to FIGS. 5A-5B, the engagement elements 56 of the engagement mechanism 48 are shown in the second position, wherein the engagement elements 56 directly contact the outer surface of the body 92 of the engagement portion 46. In particular, the edge or pointed terminal end 86 of each engagement element 56 physically contacts the outer surface 96 of the body 92 and is embedded or digs into the material of the engagement portion 46 of the rolling diaphragm syringe 20. In this manner, the engagement elements 56 are directly connected to the engagement portion 46 of the rolling diaphragm syringe 20 to allow the proximal end wall 34 rolling diaphragm syringe 20 to be pulled back or unrolled with a proximal movement of the piston 19. This embedding or digging action of the edge or pointed terminal end 86 of each engagement element 56 may be increased during proximal movement of the piston 19, particularly if the edge or pointed terminal end 86 is angled in a proximal direction relative to the engagement portion 46 so that proximal movement burrows the edge or pointed terminal end 86 of each engagement element 56 further into the engagement portion 46. The grooves or splines of the present disclosure reduces the amount of surface material on the body 92 into which the engagement elements 56 must burrow, allowing the engagement elements 56 to burrow further into the material of the body 92.

With reference to FIGS. 5C-5E, contact between the edge or pointed terminal end 86 of each engagement element 56 with the outer surface 96 of the body 92 deforms at least a portion of the body 92 and the plurality of grooves 94. For example, as shown in FIG. 5E, contact between the edge or pointed terminal end 86 of each engagement element 56 with the outer surface 96 of the body 92 may deform the body 92 by drawing excess material proximally to form the berm 98 proximal to a proximal edge 102 of the edge or pointed terminal end 86. The berm 98 may prevent skipping or dragging of the engagement elements 56 along the longitudinal length of the body 92 of the engagement portion 46 during proximal movement of the piston 19. The deformation of the engagement portion 46 of the rolling diaphragm syringe 20 due to contact with the engagement elements 56 allows the engagement elements 56 to grip the engagement portion 46. Incorporation of the grooves or splines of the present disclosure into the surface of the engagement portion 46 allows the engagement elements 56 to more readily dig into the material of the outer surface 96 of the body 92 of the engagement portion 46 thereby increasing the grip of the engagement elements 56 on the engagement portion 46. In addition to the deformation of the outer surface 96 of the body 92 of the engagement portion 46, the one or more grooves 94 may be deformed due to engagement between the edge or pointed terminal end 86 of each engagement element 56 with the outer surface 96 of the body 92. For example, the base surface and/or the pair of sides may be deformed with the deformation of the outer surface 96 of the body 92. In embodiments where the body 92 has a widened portion 65 at its proximal end, such as shown in FIG. 6B, the widened portion 65 may provide a stop surface that prevents sliding of the engagement elements 56 in an axial direction relative to the body 92. According to certain embodiments having widened portion 65, the one or more grooves 94 may be on both the body 92 of engagement portion 46 and on the widened portion 65. In other embodiments having widened portion 65, the one or more grooves 94 may be only on the body 92 of engagement portion 46 and not on the widened portion 65.

In some embodiments, the outer surface 96 of the body 92 including the one or more grooves 94 may be plastically deformed such that its shape is permanently changed after the engagement elements 56 are disengaged from the outer surface 96 of the body 92. In other embodiments, the outer surface 96 of the body 92 including the one or more grooves 94 may be elastically deformed during contact with the engagement elements 56. According to certain embodiments, after disengaging the engagement elements 56 from the engagement portion 46, the outer surface 96 of the body 92 may revert to its original shape prior to engagement with the engagement elements 56. In other embodiments, after disengaging the engagement elements 56 from the engagement portion 46, the outer surface 96 of the body 92 may remain deformed by the contact with the engagement elements 56.

While examples of a fluid delivery system and a syringe for use therefor were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1. A rolling diaphragm syringe comprising:
a proximal end having an end wall;
a distal end having an open-ended discharge neck;
a sidewall extending between the proximal end and the distal end along a longitudinal axis;
a piston engagement portion protruding proximally from a central portion of the end wall, the piston engagement portion having a central axis; and
a plurality of grooves recessed inward relative to an outer surface of the piston engagement portion and spaced apart from each other in a direction about the central axis,
wherein at least a portion of the sidewall is flexible such that:
the sidewall rolls upon itself with an outer surface of the sidewall at a folding region being folded in a radially inward direction when acted upon by an external force in a direction from the proximal end toward the distal end, and
the sidewall unrolls with the outer surface of the sidewall at the folding region being unfolded in a radially outward direction when acted upon by the external force in a direction from the distal end toward the proximal end.

2. The rolling diaphragm syringe according to claim 1, wherein the plurality of grooves are spaced apart at equal or unequal intervals along the outer surface of the piston engagement portion in the direction about the central axis.

3. The rolling diaphragm syringe according to claim 1, wherein the plurality of grooves have equal or unequal width measured along the outer surface of the piston engagement portion in the direction about the central axis.

4. The rolling diaphragm syringe according to claim 1, wherein the plurality of grooves have equal or unequal depth measured from the outer surface of the piston engagement portion in a direction toward the central axis.

5. The rolling diaphragm syringe according to claim 1, wherein each groove has a base surface and a pair of side surfaces extending from the base to the outer surface of the engagement portion.

6. The rolling diaphragm syringe according to claim 1, wherein the plurality of grooves are continuous over a longitudinal length of the piston engagement portion in a direction along the central axis.

7. The rolling diaphragm syringe according to claim 1, wherein at least one of the plurality of grooves is discontinuous over a longitudinal length of the piston engagement portion in a direction along the central axis.

8. The rolling diaphragm syringe according to claim 1, wherein an outer diameter of the outer surface of the piston engagement portion is uniform in a direction along the central axis.

9. The rolling diaphragm syringe according to claim 1, wherein an outer diameter of the outer surface of the piston engagement portion is non-uniform in a direction along the central axis.

10. The rolling diaphragm syringe according to claim 9, wherein an outer diameter of the outer surface of the piston engagement portion decreases from a distal end of the piston engagement portion toward a proximal end of the piston engagement portion.

11. The rolling diaphragm syringe according to claim 9, wherein an outer diameter of the outer surface of the piston engagement portion is greater at the distal end and proximal end than in a middle of the piston engagement portion between the distal end and the proximal end along the central axis.

12. The rolling diaphragm syringe according to claim 9, wherein an outer diameter of the outer surface of the piston engagement portion is greater at a proximal end of the piston engagement portion than along a remaining portion of the outer diameter of the piston engagement portion.

13. The rolling diaphragm syringe according to claim 1, wherein the piston engagement portion is monolithically formed with the end wall.

14. The rolling diaphragm syringe according to claim 1, wherein the piston engagement portion is configured for engagement with at least one engagement element of a piston of a fluid injector at least during movement of the piston in a proximal direction.

15. The rolling diaphragm syringe according to claim 14, wherein at least a portion of the piston engagement portion is plastically deformed when engaged with the at least one engagement element of the piston.

16.-19. (canceled)

20. A syringe assembly for a fluid delivery system, the syringe assembly comprising:
a pressure jacket having a pressure jacket distal end, a pressure jacket proximal end, and a throughbore extending between the pressure jacket distal end and the pressure jacket proximal end along a longitudinal axis; and
a rolling diaphragm syringe disposed within the throughbore of the pressure jacket, the rolling diaphragm syringe comprising:
a proximal end having an end wall;
a distal end having an open-ended discharge neck;
a sidewall extending between the proximal end and the distal end along the longitudinal axis;
a piston engagement portion protruding proximally from a central portion of the end wall, the piston engagement portion having a central axis; and
a plurality of grooves recessed inward relative to an outer surface of the piston engagement portion and spaced apart from each other in a direction about the central axis,
wherein at least a portion of the sidewall is flexible such that:
the sidewall rolls upon itself with an outer surface of the sidewall at a folding region being folded in a radially inward direction when acted upon by an external force in a direction from the proximal end toward the distal end, and
the sidewall unrolls with the outer surface of the sidewall at the folding region being unfolded in a radially outward direction when acted upon by the external force in a direction from the distal end toward the proximal end.

21. The syringe assembly according to claim 20, further comprising a movable closure for selectively enclosing at least a portion of the distal end of the rolling diaphragm syringe within the pressure jacket.

22.-40. (canceled)

41. A method for engaging an engagement portion of a rolling diaphragm syringe, the method comprising:
engaging an edge surfaces of a terminal end of one or more engagement elements of a piston with an outer surface of the engagement portion, wherein at least a portion of the outer surface has a plurality of inwardly recessed grooves thereon; and
at least partially embedding the edge surfaces of the terminal end of the one or more engagement elements into a material of the plurality of inwardly recessed grooves on at least the portion of the outer surface of the engagement portion.

42. The method of claim 41, further comprising retracting the piston in a proximal direction,
wherein retracting the piston initiates engaging the edge surfaces of the terminal end of the one or more engagement elements of the piston with an outer surface of the engagement portion.
